(19) United States
(12) Patent Application Publication
(10) Pub. No.: US 2026/0050593 A1
(43) Pub. Date: Feb. 19, 2026

(54) PROVIDING ACCESS TO CLINICAL TRIAL DATA

(71) Applicant: IQVIA Inc., Parsippany, NJ (US)

(72) Inventors: Srikanth Sankaran Iyer, King of Prussia, PA (US); Tanveer Ahmed Nasir, Chesterbrook, PA (US); Thomas I. Starr, Westbury, Wiltshire (GB); Lars Jonas Mikael Renström, Uppsala (SE); Hao Ran Laurence Lin, Holly Springs, NC (US)

(21) Appl. No.: 19/095,170

(22) Filed: Mar. 31, 2025

Related U.S. Application Data
(63) Continuation-in-part of application No. 18/956,681, filed on Nov. 22, 2024.
(60) Provisional application No. 63/562,918, filed on Mar. 8, 2024.

(51) Int. Cl. G06F 16/2453; G06F 16/28; G16H 10/20

(52) U.S. Cl. CPC G06F 16/24542; G06F 16/284; G16H 10/20

(57) ABSTRACT

Methods and systems for retrieving data from at least one database based on a query input related to a clinical trial. A system processes a received query input related to a clinical trial using a pre-trained language model neural network. The neural network generates a structured representation of the query input. The system maps a first data field of the structured representation to a first column name and maps a second data field of the structured representation to a second column name. The system generates a database query based on (i) a database schema, (ii) the first column name, (iii) the second column name, (iv) data values associated with the first data field, and (v) data values associated with the second data field. The database query specifies an operation for joining data associated with the first column name with data associated with the second column name.

### CLAIM OF PRIORITY

This application is a continuation-in-part of U.S. patent application Ser. No. 18/956,681, filed on Nov. 22, 2024, which claims priority under 35 USC § 119(e) to U.S. Patent Application Ser. No. 63/562,918, filed on Mar. 8, 2024, the entire contents of both of which are hereby incorporated by reference.

### BACKGROUND

Clinical trials generate data that inform medical research, regulatory approval, and healthcare practices. Access to clinical trial data is essential for a variety of stakeholders to evaluate treatment effectiveness and safety. The process of obtaining and analyzing clinical trial data can be hindered by complex data formats, data segmented across large repositories, and inconsistent data standards across studies. Efficient solutions for accessing and organizing clinical trial data can increase efficiency of data retrieval and analysis.

### SUMMARY

The systems and techniques described here relate to accessing clinical trial data and generating ad hoc analysis of the accessed clinical trial data. By leveraging generative artificial intelligence (GenAI) systems, knowledge graphs in relation to database schemas, and domain expertise, clinical trial data can be accessed, analyzed, and presented to a user in response to a natural language query.

These methods include generating a structured representation of the natural language query by a GenAI system. In some cases, the natural language query includes one or more terms that can define the parameters of database query in terms of data values and columns names stored in a database. Based on the structured representation generated by the GenAI system in response to receiving the natural language query, a system can generate a database query with a rules-based approach based on the database schema to access relevant data from the database according to the language present in the natural language query.

In some cases, the methods include feedback loops based on storing and updating feedback data that can include reviewed generated database queries, examples, alternative column names, and scored outputs from the GenAI system. The stored feedback data can be used to refine future executions of the GenAI system and database query generation. In some implementations, the feedback data includes reviewed data by a domain expert.

The subject matter described in this specification can be implemented in particular embodiments to realize one or more of the following advantages. Techniques are described for implementing a method for accessing clinical trial data. In some cases, the techniques include a translation of a natural language query into executable code that results in a retrieval of data that more accurately reflects the objective of the query in comparison with alternative approaches. Additionally, the techniques allow for a near real-time (e.g., within a time frame associated with processing of data as described in this specification) and ad-hoc (e.g., customizable and in-response to particular user requests) delivery of analytical insights and reports. Furthermore, the techniques allow for users to receive customizable analytical insights with fewer database queries due to the technique of generating executable code that delivers the desired analytical insights without human intervention, resulting in a usage of fewer computational resources and less data transmission bandwidth.

Additionally, the insights (e.g., data tables, metrics, charts, reports, etc.) generated by the system can be integrated into other operational tools, such as workflow and audit trail capabilities related to clinical trials. The system provides a convenient user experience, e.g., a user can request clinical data in common language and the computer performs the data query as if the computer can understand the user as another human does. The system also makes it easy for the end user to access information they need without having to know exactly where to find it in a system that includes multiple databases and dashboards (e.g., in which dashboard or part of data set), saving time and removing the need to manually manipulate and merge different data sets. Additionally, the GenAI system can be tuned with user, company, and/or domain-specific natural language (e.g., proprietary/internal key phrases to ask specific questions and to utilize specific abbreviations and acronyms) and intellectual properties (e.g., algorithms to evaluate risk).

The methods described here standardize and facilitate preparation of reports that are often otherwise performed manually by operational roles and personnel that focus on analytics and/or operational oversight. For instance, the GenAI system enables reports/calculations to be prepared using standardized coding and proper statistics (as defined by parameters of the GenAI and rules-based systems determined by domain experts), in a way that is sharable across users and across datasets.

Furthermore, the methods described here facilitate a generation of insights from data stored in a localized database (e.g., a database with data fields, data values, and data contexts associated with a particular entity, e.g., a company, organization, etc.). Accommodating localized databases allows for a more flexible approach to converting a natural language prompt into high-level data insights extracted from a localized database. The particular methods of accommodating localized data for insight generation and database code generation allows for the generation of accurate database code (e.g., database queries) with fewer computational resources. The system can generate accurate database code with fewer language model inferences due to a combination of syntactic entity extraction, which relies on keyword matching between a user query and database entities (e.g., column names), and semantic entity extraction, which relies on LLM inferences to identify relevant entities in the user query.

In one aspect, a system for retrieving data from at least one database includes at least one processor and a memory storing instructions that, when executed by the at least one processor, cause the at least one processor to perform operations that include processing a received query input related to a clinical trial using a pre-trained language model neural network. The neural network is configured to generate data indicative of a structured representation of the query input, in which the structured representation includes multiple data fields and corresponding data values. The operations include mapping a first data field of the structured representation to a first column name of a first table of a relational database, in which the relational database is characterized by a database schema and mapping a second data field of the structured representation to a second column name of a second table of the relational database. Furthermore, the operations include generating a database query based on the database schema, the first and second column names, and the respective data values associated with the first data field and the second data field. The database query specifies an operation for joining data represented in the data column associated with the first column name with data represented in the data column associated with the second column name.

In some implementations, the operations include executing the generated database query, in which an output of the executed query is a resulting data table and displaying, on a user interface, a visual representation of the resulting data table. In some implementations, the operations include generating executable code by a pre-trained language model neural network for analyzing the resulting data table.

In some implementations, the relational database includes at least one calculated table, in which the calculated table includes data from at least two tables of the relational database. In some implementations, the calculated table is determined based on one or more rules associated with received domain expertise.

In some implementations, the operations include generating an embedded representation of a data field of the structured representation, generating an embedded representation of at least a portion of a column name associated with a table of the relational database, and determining a similarity metric between the data field and the portion of the column name, the similarity metric based on an overlap of the embedded representations.

In some implementations, at least one column of at least one table of the relational database is characterized by a corresponding one alternative column name, the alternative column name different from the column name.

In some implementations, the operations include receiving feedback indicative of an accuracy of the mapping of the first data field to the first column name and updating at least one alternative column name of a column of a table of the relational database.

In some implementations, the operations include (i) determining, by a syntactic entity extraction agent, one or more localized entities in the query input, in which the syntactic entity extraction agent performs one or more search techniques to match a phrase in the query input with localized entities of a localized database, (ii) determining, by a semantic entity extraction agent, one or more entities in the query input, in which the semantic entity extraction agent processes the query input and schema information of the localized database with a large language model, (iii) combining the localized entities determined by the syntactic entity extraction agent and the entities determined by the semantic entity extraction agent to generate a combined set of entities, and (iv) generating, by the large language model, a database query that includes query statements associated with the combined set of entities.

In some implementations, the operations include parsing the generated database query to determine associated database query statements, the statements including select parameters, filter parameters, and grouping parameters, and generating a final database query with a rules-based approach based on the determined database query statements.

In some implementations, the operations include determining a subset of data from the localized database by querying the localized database with the generated final database query.

In some implementations, the operations include displaying the subset of data on a user interface.

In some implementations, the one or more search techniques include exact string matching and fuzzy string matching.

In some implementations, the semantic entity extraction agent processes the output of the syntactic entity extraction agent with a large language model to generate a database query.

In some implementations, the schema information includes data field names, data field values, and data types pertaining to the data stored in the localized database.

In some implementations, the operations include retrieving data indicative of user access settings, wherein the user access settings include data indicative of an authentication for displaying the determined subset of data on the user interface.

In some implementations, the entities determined by the semantic entity extraction agent are highly represented in training data using to train the large language model.

In some implementations, the entities determined by the syntactic entity extraction agent are not highly represented in training data used to train the large language model.

In one aspect, a method for retrieving data from at least one database includes processing a received query input related to a clinical trial using a pre-trained language model neural network. The neural network is configured to generate data indicative of a structured representation of the query input, in which the structured representation includes multiple data fields and corresponding data values. The method includes mapping a first data field of the structured representation to a first column name of a first table of a relational database, in which the relational database is characterized by a database schema and mapping a second data field of the structured representation to a second column name of a second table of the relational database. Furthermore, the method includes generating a database query based on the database schema, the first and second column names, and the respective data values associated with the first data field and the second data field. The database query specifies an operation for joining data represented in the data column associated with the first column name with data represented in the data column associated with the second column name.

In another aspect, one or more non-transitory computer readable media store instructions that, when executed by at least one processor, cause the at least one processor to retrieve data from at least one database based on a query input related to a clinical trial by performing operations that include processing a received query input related to a clinical trial using a pre-trained language model neural network. The neural network is configured to generate data indicative of a structured representation of the query input, in which the structured representation includes multiple data fields and corresponding data values. The operations include mapping a first data field of the structured representation to a first column name of a first table of a relational database, in which the relational database is characterized by a database schema and mapping a second data field of the structured representation to a second column name of a second table of the relational database. Furthermore, the operations include generating a database query based on the database schema, the first and second column names, and the respective data values associated with the first data field and the second data field. The database query specifies an operation for joining data represented in the data column associated with the first column name with data represented in the data column associated with the second column name.

The details of one or more embodiments of the invention are set forth in the accompanying drawings and the description below. Other features, objects, and advantages of the invention will be apparent from the description and drawings, and from the claims.

### BRIEF DESCRIPTION OF THE DRAWINGS

FIG. 1 illustrates an example system for accessing clinical trial data.

FIG. 2 illustrates an example system for updating a data store based on collected feedback.

FIG. 3 illustrates an example system for generating a database query.

FIG. 4 illustrates an example system for generating a database query and an ad hoc data analysis based on an interaction with user at a user interface.

FIG. 5 illustrates an example system for interpreting a user query with a GenAI system.

FIG. 6 is a flow diagram of an example process for accessing clinical trial data.

FIG. 7 illustrates a system for generating a database query based on a user query.

FIG. 8 illustrates a system for generating a database query for a localized database.

FIG. 9 is a flow diagram of an example process for generating a database query based on a user query.

FIG. 10 is a graphical representation of a comparison between an implementation of a default GenAI system and a GenAI system that accesses a localized database.

Like reference numbers and designations in the various drawings indicate like elements.

### DETAILED DESCRIPTION

The systems and techniques described here relate to accessing and generating ad hoc analyses of clinical trial data that is stored in one or more databases using a natural language query. In some cases, generative artificial intelligence (GenAI) systems can transform a natural language query (e.g., a question or a prompt) into a format that can be interpreted as and/or transformed into multiple database queries.

These methods include a generation of a structured representation of a natural language query that includes relevant data fields along with values, or ranges of values, that can be found in a database. For example, a natural language query from a user can include a requested number of overdue action items from the past three weeks in relation to a particular clinical trial that is operating in a particular country. As such, a user may input a query like, “How many overdue action items (overdue days greater than 21 days) are there for clinical trial XYZ in the United States?”. A structured representation of this query can include a data structure that includes the ordered pairs of (overdue action items, count( )), (overdue action items overdue days, >21 days), (country, United States), (clinical trial, XYZ). In some implementations, a GenAI system processes the input query along with supplementary contextual information (e.g., examples from past prompts, feedback data, etc.) to generate the structured representation. The GenAI system converts unstructured natural language into the structured representation that includes domain-relevant data fields and corresponding values.

Based on the structured representation of the natural language query, a system can match the determined fields to one or more tables of a relational database. In some implementations, the system can generate a database query (e.g., a SQL query), that joins data from multiple tables to access data that is relevant to answering the question of the input query. In some implementations, the system access and joins tables from multiple distinct databases.

The system can execute the generated database query to determine a dataset that can be used to address the specific question and/or prompt of the natural language query. For example, a system can process the dataset to generate a plot, chart, list of metrics, etc., depending on the context of the natural language query.

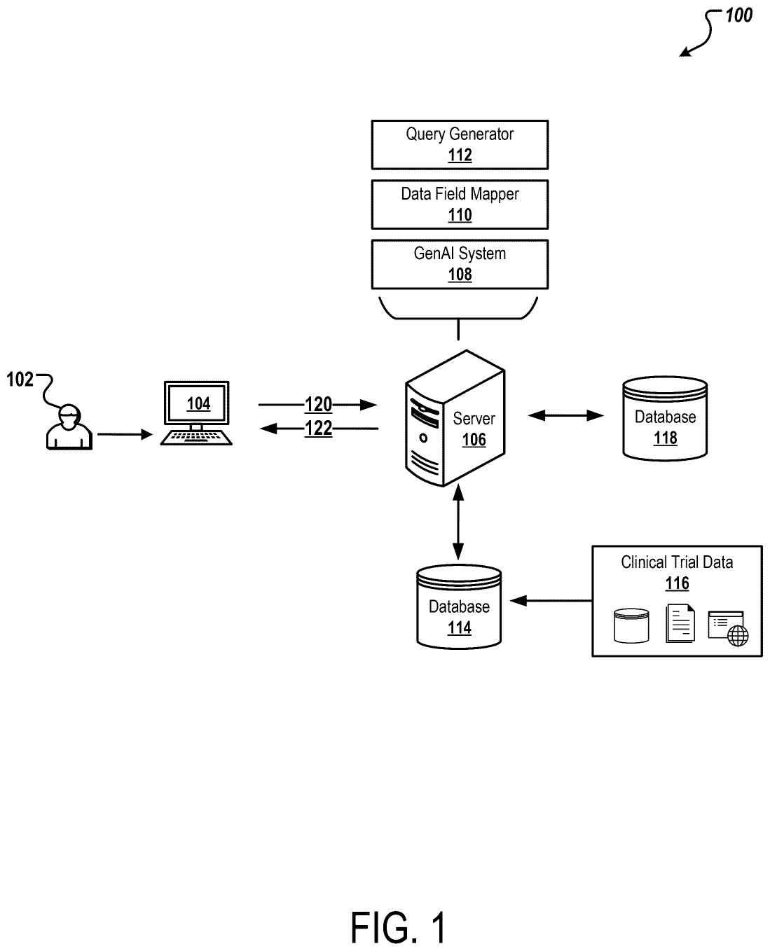

FIG. 1 illustrates an example system 100 for accessing clinical trial data. The system 100 depicts a user 102 that interacts with a user interface 104. In some implementations, the user interface 104 is implemented on a client device (e.g., laptop, mobile device, etc.) and can receive a natural language query 120 from the user 102 and transmits the query over a communication channel to a server 106. In some implementations, the server 106 performs one or more data processing tasks. For example, the server 106 can perform operations of a generative AI system (GenAI system 108), a data field mapper 110, and a query generator 112. Other operations related to data processing, data communications, user access and authentication, and others can be implemented by the server 106. In some implementations, user access and authentication data includes data values indicative of an authentication for displaying one or more data fields on a user interface for viewing by a particular user or group of users.

In some implementations, operations implemented by the server 106 are implemented by more than one server. In some implementations, the server 106 accesses one or more databases. For example, the server 106 can access a database 114 of clinical trial data and a database 118 of example data in relation to queries received from the user interface 104 and responses provided to the user interface 104. In some implementations, the server 106 is communicatively coupled with one or more other servers that perform additional data processing tasks, data acquisition tasks, etc. via communication channels including an application programming interface (API).

The server 106 performs operations of the GenAI system 108 which can include processing an embedded representation (i.e., a numerical, machine-readable representation) of the query 120 and additional contextual information (e.g., examples, instructions, etc.) from the user interface 104. In some implementations, the GenAI system 108 includes a large language model (LLM). In some implementations, the GenAI system 108 includes a machine learning model that processes the query 120 and generates a structural representation of the query 120.

In some implementations, an LLM of the GenAI system 108 is a neural network machine learning model with a particular neural network structure. The particular neural network structure can include a transformer network that includes any or all of a recurrent layer (e.g., data processing layer that captures relationships between words), an embedding layer (e.g., data processing layer that converts words into machine-readable numerical vectors), feed forward layer (e.g., data processing layer that transforms embedded representations), and attention layer (e.g., data processing layer that considers the positions and relationships of words in a sentence in relation to other words in the sentence). In some implementations, the function of an LLM is to predict a “next word” following an input word sequence. In other words, an LLM can process an input sequence of words and provide a sequence of subsequent words that have a certain probability of following the input sequence based on a set of training data used to train the LLM.

In some implementations, the GenAI system 108 implemented on the server 106 can process the query 120 along with supplementary data that is stored in the database 118 and accessed by the GenAI system 108 via a data access protocol (e.g., SQL queries, key-value pairs, etc.) implemented by the server 106. The supplementary data can include any or all of examples of prompt-response pairs generated and/or reviewed by domain experts, additional data related to the query 120 (e.g., background information about a topic, a particular academic journal article, etc.), and particular instructions for the GenAI system 108 to follow with respect to text formatting, response style, analysis instructions, etc. In a general sense, the approach of processing the query 120 with supplementary information stored in the database 118 is referred to as prompt engineering or prompt design, in which the prompt is processed by the LLM of the GenAI system 108 and includes additional context that may be outside of the context of the training data used to train the LLM. In some implementation, a system prompt is provided to the LLM of the GenAI system 108 before processing a user query, in which the system prompt represents grounding information (e.g., general guidelines, instructions, response style, etc.),

In some implementations, examples of queries and GenAI responses are collected and stored as constructive examples in the database 118 (or a file, table, vector store, etc.) that can be queried at a later time (e.g., via a search function based on Euclidean distance or cosine similarity) to retrieve relevant example responses for a particular prompt.

In the context of this disclosure, the GenAI system 108 processes a prompt that includes the query 120 along with instructions to generate a structured representation of the query 120. The structured representation includes one or more key-value pairs in which a “key” of a key-value pair corresponds to a column (data field) of a table of the clinical trial database 114. A “value” of a key-value pair corresponds to a target value or range of values of the data represented in the identified column of the table of the clinical trial database 114. In some implementations, the clinical trial database 114 includes multiple databases hosted on multiple servers, in which each database includes multiple tables. In relation to the example provided above, the GenAI system 108 can transform the query “How many overdue action items (overdue days greater than 21 days) are there for clinical trial XYZ in the United States?” into a structured representation that can be visualized by the table:

| Field Name | Values |
|---|---|
| Overdue Action Items | Count( ) |
| Overdue action item overdue days | Over 21 days |
| Country | United States |
| Clinical Trial | XYZ |

The structured representation includes a first column that describes, in natural language, a field associated with the query 120. The structured representation includes a second column that describes a value or range of values that are found in the query 120 and correspond to a respective field. In addition to extracting fields from the query 120, the GenAI system 108 can process mathematical requests. For example, the GenAI system 108 can interpret a difference between a phrase “>=21 days” and “>=21 days but <=60 days.” Furthermore, the GenAI system 108 can also interpret phrases that include dates, e.g., “>1 Feb. 2025,” and “>1 Feb. 2025 and <28 Feb. 2026.”

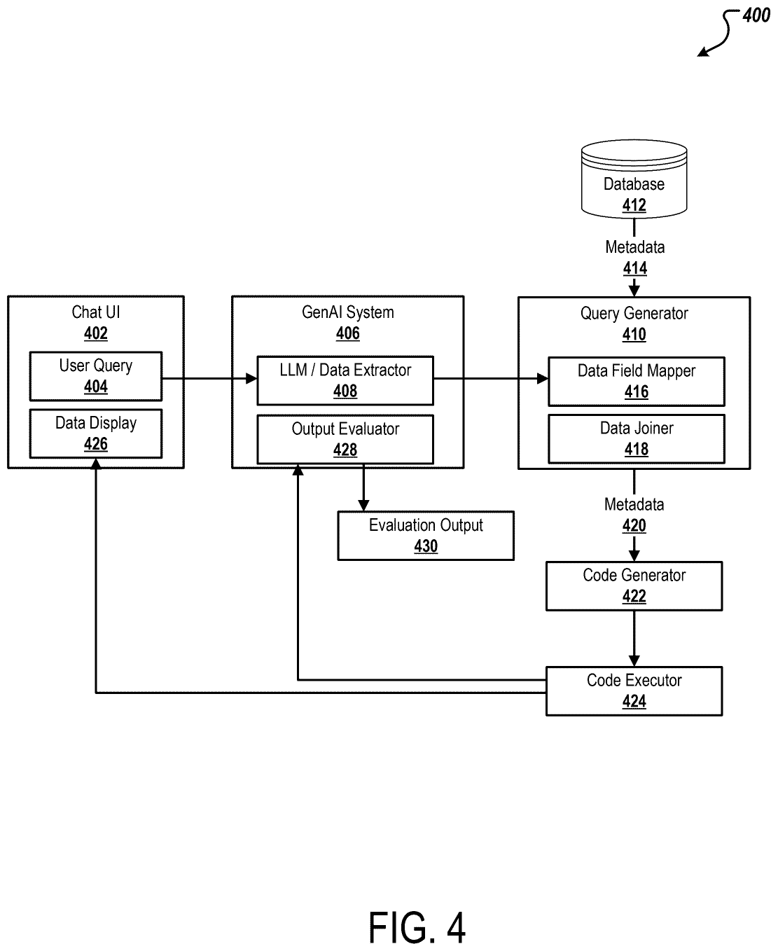

In addition to the operations associated with the GenAI system 108, the server 106 implements operations associated with the data field mapper 110. The data field mapper 110 maps the fields (Field Name) of the table depicted above to column names in the clinical trial database 114. In some implementations, the data field mapper 110 executes operations associated with one or more machine learning models to predict a likely column in the database 114 for each field in the generated structured representations. In some other implementations, the data field mapper 110 executes operations related to determining string similarity between the generated fields of the structured representation and column names of the database 114. In some implementations, as described below in relation to FIG. 4, each column and/or table of the database 114 includes a name (e.g., column name) and additional alternative names commonly used to describe the data represented in the column and/or table. In some cases, the alternative names are determined based on domain expertise and/or previous queries of the system.

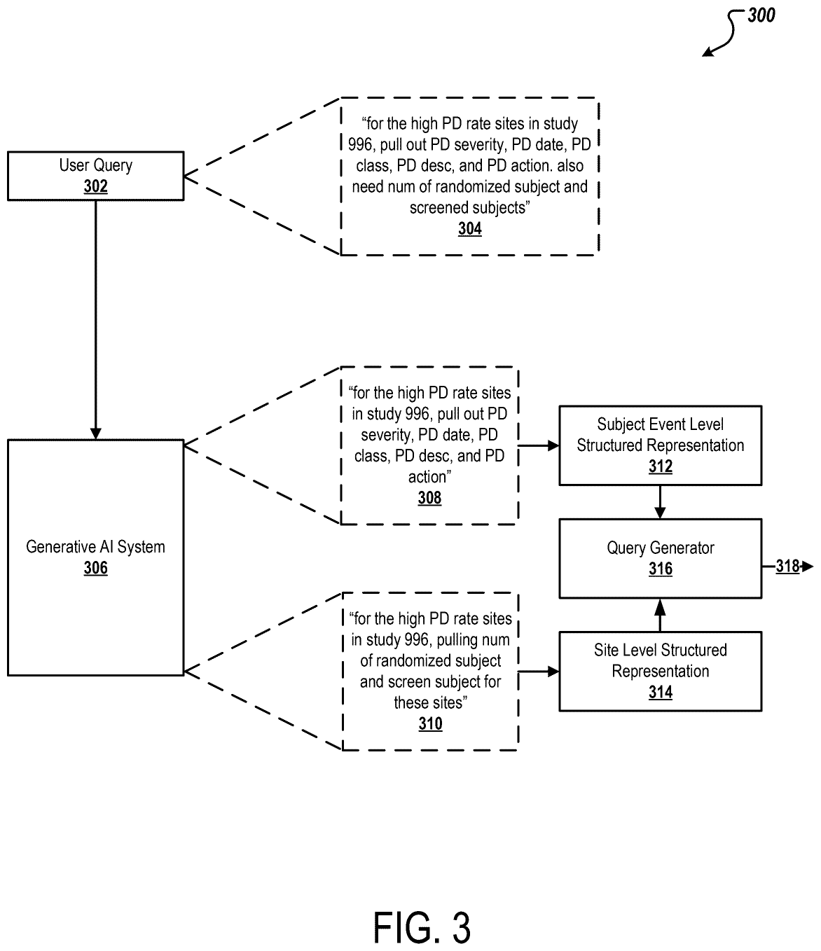

The server 106 implements operations associated with the query generator 112. The query generator 112 processes the identified data fields from the data field mapper 110 along with the data field values (represented in the second column of the table above) to generate a database query in relation to the database 114. In some implementations, the database 114 is described by a database schema, described in more detail in relation to the description of FIG. 3. In some implementations, the query generator 112 is a rules-based process that generates a database query deterministically based on the database schema and the generated structured representation.

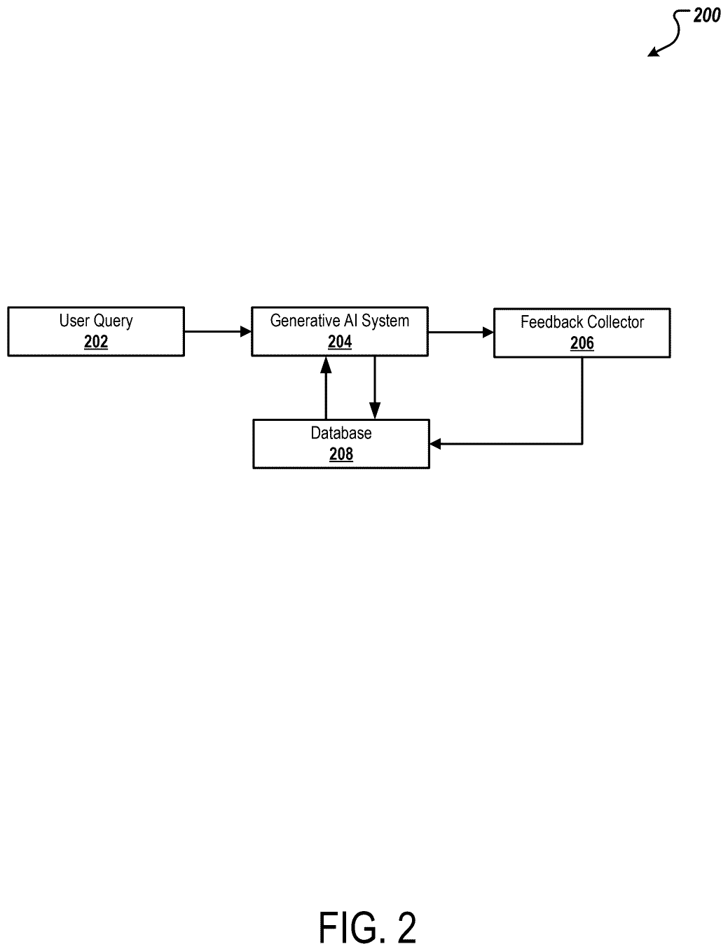

As described in relation to the following description of FIG. 2, the system can include one or more feedback loops that can iteratively improve the accuracy of the generation of structured representations by the GenAI system 108, the matching efficiency and accuracy of the data field mapper 110, and the accuracy of database query generation by the query generator 112.

FIG. 2 illustrates an example system 200 for updating a database 208 based on collected feedback. The system 200 includes a user query 202 that is received and processed by a generative AI system (GenAI system 204). A feedback collector 206 executes one or more feedback collection processes (e.g., displaying outputs on a user interface and receiving feedback through the user interface, generating feedback through an automated system via a machine learning model, etc.) and stores the collected feedback in the database 208. The generative AI system 204 accesses data stored in the database 208 as examples, to determine particular rules, to identify particular patterns, etc., and processes the accessed data along with data indicative of the received user query 202 (e.g., a prompt).

In some implementations, the database 208 stores data as a relational database, key-value pairs, data files, embedding vectors in a vector store, encrypted data files, or any other means of digitally storing data.

In some implementations, the feedback collector 206 gathers user feedback of GenAI outputs (e.g., correctness of human language interpretation, correctness of finding the data variables in the source databases, interpreted chart/slide title, labels, and descriptions) for progressive model improvements (e.g., adjusting training data, adjusting data for fine-tuning, adjusting GenAI prompts, adding/removing examples from the database 208 provided to the GenAI system 204). In addition, in some implementations, users are guided by the GenAI system 204 (e.g., via a chat interface) to make a particular type of query (e.g., based on the past working examples and query library). Furthermore, the approaches described here enable knowledge (query) transfer from one study to another study under similar conditions and shares existing queries among team members within a study to avoid duplicate works among different team members.

The process of accessing data from the database 208 and including the accessed data in a prompt that is processed by the GenAI system 204 is commonly referred to as retrieval augmented generation (RAG). RAG is a technique that combines information retrieval with text generation. The technique is often implemented as a part of a solution for a task that requires extensive or specific knowledge that is not stored in the LLM of the GenAI system 204 itself, such as question-answering systems or providing detailed information about specialized topics or custom datasets.

In the example use cases related to this specification, the RAG processes includes augmenting a prompt that includes the user query 202 and potentially hard-coded instructions for the GenAI system 204 to follow with examples of inputs/outputs along with feedback data collected by the feedback collector 206 and stored in the database 208. As users interact with the system that includes the GenAI system 204, the feedback collector 206 can collect and store more feedback data and reviewed examples to store in the database 208 for access by the GenAI system 204 in order to improve the quality and accuracy of the generated outputs.

As described below in relation to FIG. 3, the operations of the GenAI system 204 can be understood as breaking apart a particular user query into multiple different questions that can be answered by accessing data from multiple databases and/or database tables.

FIG. 3 illustrates an example system 300 for generating a database query. The system 300 includes a GenAI system 306 that processes a user query 302. The user query 302 is a natural language query that is related to a question or a prompt that is related to data in a particular database.

An example query input 304 that represents the user query 302 of the example system 300 is, “for the high PD rate sites in study xyz, pull out PD severity, PD date, PD class, PD desc, and PD action. Also need num of randomized subject and screened subjects.” In this example, a user submits the example query input 304 that includes grammatical mistakes, incomplete sentences, non-standardized data fields, and multiple questions. Traditionally, a person (e.g., a developer, database engineer, analyst, etc.) receives the example query input 304, analyze what the user wants, and generates a database query that potentially accesses data from multiple databases across multiple tables, join all of the data in a way that preservers consistency across data fields, and generates various analytical outputs to be provided to the user that submitted the user query 302. The techniques described in this specification provides an automated solution to replace the above manual task completion.

In a general sense, the GenAI system 306 is operable to decompose the user query 302 into one or more structured representations of the user query 302 that can be analyzed and executed by a system that generates and executes database queries. The particular method of generating the structured representations is carried out by the LLM of the GenAI system 306. The LLM is trained on general language data, and can be prompted by specific tasks, examples, instructions, etc., in order to generate the structured representations in relation to the user query 302. To illustrate how a system like the GenAI system 306 generates the structured representations, it is useful to consider how an LLM may decompose a query into multiple tasks related to determining a response based on data present in database tables. The description of how the LLM decomposes a query into multiple tasks is an illustrative example of how a structured representation can be generated. In many cases, the LLM operations are executed “under the hood” of the trained neural network and the operations can only be inferred based on an analysis of the input and output data.

The system 300 provides an illustrative example of how the GenAI system 306 can implement operations of an LLM to analyze the user query 302 and determine one or more structured representations of the user query 302. In the context of the example system 300 and the example query input 304, the GenAI system 306 determines two discrete tasks. A first task 308 includes a query, “for the high PD rate sites in study xyz, pull out PD severity, PD date, PD class, PD desc, and PD action.” A second task 310 includes a query, “for the high PD rate sites in study xyz, pulling num of randomized subject and screen subject for these sites.” The GenAI system 306, based on the internal LLM that is trained on general language tasks, identifies the first task 308 and the second task 310 that can be addressed by forming and executing database queries directed towards two separate tables of a particular relational database. The trained LLM can identify patterns in language and understand likely meanings to abbreviations and user intention (e.g., the LLM can determine that “PD” is referring to “protocol deviations” in the context of clinical trials). In a general sense, the LLM need not have information of the specific relational database, but in some cases, the GenAI system can include instructions to the LLM that may include high-level information about a relational database (e.g., column names, key names, etc.), or an indication that the LLM should generate discrete tasks from the user query 302, in which each discrete task is related to a single table of a particular database.

The first task 308, identified by the GenAI system 306, is associated with a generation of a subject event level structured representation 312. The representation 312 includes data about particular subject events related to a clinical trial. By processing the representation 312 with a data field mapper (e.g., the data field mapper 110), the representation 312 is relevant to data from a table from the database that represents protocol deviations (e.g., “PD tbl”). The second task 310 is associated with a generation of a site level structured representation 314. By processing the representation 314 with a data field mapper (e.g., the data field mapper 110), the representation 314 is relevant to data about particular site-level indications related to a clinical trial. The data field mapper identifies the representation 314 to be related to data from three tables from the database that represent (i) study site information (e.g., “study site tbl”), (ii) study subject information (e.g., “study subject tbl”), and (iii) subject status information (e.g., “subject status tbl”). For example, the GenAI system 306 along with a data field mapper, based on the general language understanding of the LLM and potentially examples from an examples database, as described in relation to FIG. 2, can identify the first task 308 to be related to a database table that may include information about protocol deviations in relation to particular subjects of a clinical trial. Additionally, the GenAI system 306 along with a data field mapper identifies the second task 310 to be related to multiple tables that may include information about site-level information regarding subject classifications (e.g., random subjects, screened subjects, etc.). The methods for determining which tables and which columns are related to a particular task (e.g., tasks 308, 310) via a data field mapper are described in relation to FIGS. 1, 4, and 5.

In some implementations, the data depicted in the representations 312, 314 are mapped to one or more column names of a relational database (via a data field mapper), in which keys of respective tables of the relational databases relate data stored in each table. In some implementations, a database schema defines the organization, structure, and constraints of the data within the relational database. The database schema serves as a reference to the architecture of the database, outlining how tables, relationships, views, indexes, and other database objects are related. In some cases, a database schema includes table definitions (e.g., the structure of the table including its columns, data types, and other constraints), relationships (e.g., a definition of how tables are related to each other through foreign keys), constraints (e.g., rules for maintaining data integrity and accuracy, such as primary keys and default values), indexes (e.g., definitions of indexes on columns to optimize search and retrieval performance within the database), and views/stored procedures (e.g., virtual tables derived from queries and/or reusable code/functions that represent complex database logic).

In some implementations, based on the representations 312, 314 a query generator 316 generates one or more database queries 318 (e.g., SQL queries) to access relevant data stored in a relational database. In some implementations, the query generator 316 accesses a knowledge base that includes domain expertise, example prompts, and other rules-based functions for mapping structured representations of a user query to a database query. The knowledge base can include a knowledge graph that stores information about database tables, column structure, joints, and meta information about the stored data. In some implementations, the query generator 316 includes one or more machine learning models.

In some implementations, the query generator 316 includes one or more rules-based algorithms for generating database queries. For example, a rues-based algorithm for generating database queries can be implemented by a Python script that processes information from the knowledge graph (e.g., column information and table joint information), to turn the representations 312, 314 into a series of executable database queries.

In some implementations, the query generator 316 includes a sequence of functions (e.g., database query executions) for determining the database queries 318. For example, based on the site-level structured representation 314, the query generator 316 can determine that the requested data includes a “study ID” (xyz), a “siteID”, and a “num of rand subj per site.” To determine a database query to address the second task 310, the query generator 316 can implement a first sub-function: “search tbls containing the data “study, site, #ofActiveSubj”->a smallest list of tables.” The first sub-function searches all of the tables in the database that includes the mentioned column names.

The query generator 316 can implement a second sub-function: “calculation table joints->slightly longer list of tbls & joint info.” The second sub-function includes an implementation of joining tables that are determined as part of the execution of the first sub-function.

The query generator 316 can implement a third sub-function: “check look-up values->passing the “xyz” to protocol_num@study_tbl->“xyz”.” The third sub-function implements a query of the “study_tbl” in which the “protocol_num” is represented by “xyz”. The output of the third sub-function represents all data of joined table that is associated with the requested study.

The query generator 316 can implement a fourth sub-function: “pulling all virtual tables for #ofActiveSubj calculation.” The fourth sub-function accesses data from virtual tables that are defined by a subject matter expert to represent data related to a “number of active subjects”. The identified virtual table is a composite table that can be saved, based on a pre-defined calculation, and accessed as if it were a standard database table.

The query generator 316 can implement a fifth sub-function: “gen Oracle-SQL.” The fifth sub-function generates executable SQL code for querying the identified virtual table based on the data from the site-level structured representation 314. In some implementations, the query generator 316 implements standardized procedures for generating database queries. For example, the query generator 316 can generate database queries with a pattern like, “with [stored executable SQL code from a virtual table] select {cols} from {tbls} where {joints} {cond}.” The variables included in the pattern (e.g., {cols}) can be substituted for values found in the structured representations 312, 314 and by rules defined in the virtual tables and/or database schema.

In some implementations, the query generator 316 accesses example executable code as generated and/or reviewed by a domain expert. The query generator 316 can access a database of executable code and perform similarity analyses and/or filtering of the code itself and/or metadata related to the code (e.g., description of the code, expected inputs/outputs, database schema, among others) to determine similar code relevant to a particular task.

In some implementations, the query generator 316 implements default decisions in relation to the generation of executable code based on the structured representations. In some cases, the default decisions are determined by one or more domain experts. For example, if a structured representation does not include a timing-related field (e.g., “in the last year”, “in the last week”, etc.), a default timing can be determined by a domain expert to be a pre-determined period of time (e.g., a week, month, year, etc.). In some implementations, the default decisions are determined based on an analysis of previous generations of executable code.

In some implementations, the query generator 316 implements a process that includes one or more steps implemented by a GenAI system. In some other implementations, the query generator 316 is entirely implemented by a GenAI system.

FIG. 4 illustrates an example system 400 for generating a database query and an ad hoc data analysis based on an interaction with user at a user interface. The system 400 includes a user interface with a chat user interface (chat UI 402). A user interacts with the chat UI 402 to submit prompts, receive responses (e.g., from a virtual assistant, chatbot, etc.), and to respond to the received responses. In general, the user submits a user query 404 via the chat UI 402. In some implementations, the user submits multiple instances of the user query 404 as part of an ongoing dialogue (e.g., chatting with a chatbot).

A GenAI system 406 receives and processes the user query 404 that is received by the chat UI 402. The GenAI system 406 includes at least one large language model (LLM) that operates as an LLM/Data extractor 408. In general, the LLM of the LLM/Data extractor 408 is operable to perform a variety of tasks related to natural language processing (e.g., predicting a next word in a sequence of words). The LLM/Data extractor 408 includes an LLM that is configured to extract data that is relevant to a particular domain (e.g., clinical trials) present in the user query 404. In some implementations, the LLM/Data extractor 408 is prompted with specific prompts related to the particular domain, provided relevant examples, and/or provided domain-specific instructions. The database of examples, prompts, and instructions are accessed by the GenAI system 406 via database connections, application programming interfaces, stored digital files, etc.

In some implementations, the user query 404 represents a question that can be answered with one or more data visualizations or data tables. In some cases, the data represented by the one or more visualizations or data tables are stored in a database. In some cases, the GenAI system 406 can generate a query, based on the content of the user query 404, to be executed to access the data from the database. In the present embodiment, the GenAI system 406 generates a structured representation of the data included in the content of the user query 404, as described in relation to the previous figures.

In some implementations, the GenAI system 406 interacts with a user via the chat UI 402 with multiple responses and a series of questions and answers to increase a probability that the generated structured representation represents the user query 404 accurately.

A query generator 410 receives and processes the structured representation generated by the GenAI system 406. The query generator 410 performs operations to convert the structured representation into an executable code, as described in relation to the query generator 316 of FIG. 3. For example, a data field mapper 416 maps the fields of the structured representation to column names and table names of a particular database (or databases). In some implementations, the structured representation includes a first data field and a value or value range that correspond to the first data field.

In some cases, the identified first data field of the structured representation does not match a column or table name of the database. For example, the first data field can be “country” and the corresponding value can be “United States”. Although a particular table might have a column named “country”, other tables might have column names like “site country”, “patient country”, etc. To determine a column of data stored in the database that corresponds to “country”, the data field mapper 416 can implement one or more probabilistic “string matching” techniques that match the first data field to the column names and table names of the database. In this case, “probabilistic” means a match with a particular likelihood of being correct.

In some implementations, each table includes a first table name and a corresponding description that includes alternative versions of the table name. Similarly, in some implementations, each column of each table of the database can include a description, or alternative column names, that include other possible variations of how one might describe the data represented in the column. In some cases, the names of data (e.g., column names) in sources are technical term abbreviations (much like code names), which are often not very useful. In some examples, alternative names (e.g., with descriptive language) are inserted into each actual data name when building the meta information about the data. Meta information of data also includes expected data type/format (as characters, as numbers, or as dates), NA handling method (what to do when there is no value), data source table, and database name. The alternative names of data can be encoded to numeric representations (e.g., embedded representations) with an encoder neural network model. Meta-information and alternatives can be updated when a database changes or a new language and/or term is used to describe a particular data. The numeric representations of both requested data variable (e.g., a column name) and data alternative names (e.g., alternative names for a column) are compared, e.g., by Euclidean distance or cosine similarity. For each requested data variable, the best matched alternative name is found and then traced back to the actual data name in the structured representation and corresponding meta information. If the best matched name is wrong, as identified during a feedback process as described in relation to FIG. 2, the alternative names list is updated with the actual data name in the meta information to increase the accuracy of match (e.g., without needing to retrain the encoder model and/or the GenAI system).

In addition to meta information, a separate database can store information about how data tables in a database can be joined. For example, joining information can include information like “Study ID in table A @ DB1 is joint by PROT_NUM in table X @ DB2.” Joining data tables from the same database is straight-forward and is implemented by executing a single query. Joining data tables from different databases (e.g., subject data is in a first database, and protocol deviation is in a second database) involves two queries, one for each database. Data joining will proceed after data are retrieved from both databases.

The data field mapper 416 can implement an exact matching technique in which a match between the first data field and a column name is identified if there is an exact match between the first data field and one or more of a column name or alternative column name.

The data field mapper 416 can implement approximate matching (i.e., fuzzy matching). Approximate matching techniques include a technique based on a Levenshtein distance, also known as edit distance, in which the edit distance measures a minimum number of single-character edits (insertions, deletions, or substitutions) required to transform one string into another. Approximate matching techniques can also include a technique based on Hamming distance which counts a number of differing characters between to strings of the same length, a Jaccard similarity which is a similarity metric that compares an intersection over a union of character n-grams (substrings of length n) or sets of words, or a Cosine similarity which represents strings as vectors and measures a cosine of the angle between them. The data field mapper 416 can implement probabilistic matching based on phonetic algorithms that encode words by their sounds or wildcard matching based on regular expressions.

In some implementations, the data field mapper 416 generates an embedded representation of the first data field. The mapper 416 can determine a similarity metric between the first data field and a portion of the column name, in which the column name can include a description that includes multiple alternative column names associated with the column. The similarity metric is determined by calculating the overlap between the embedded representation of the first data field and embedded representations of each of the alternative column names and/or an embedded representation of the column name.

Additional string matching techniques can be implemented by the data field mapper 416 to identify matches between the first data field present in the structured representation generated by the GenAI system with the table names, column names, and/or alternative names of each.

After the query generator 410 identifies relevant tables and columns that correspond to the data fields of the structured representation, the query generator 410 can generate an executable database query to retrieve the data stored in a database 412. In some implementations, the query generator 410 first receives database metadata 414 that corresponds to the database 412. In some implementations, the database metadata 414 includes database schema of the database 412. The database query generator 410 executes the executable database query to access a subset of data stored in the database 412. In some implementations, a data joiner 418 combines data from multiple tables and/or columns into a single data table with associated metadata 420.

In some implementations, the query generator 410 receives the data fields of the structured representations (i.e., outputs of the LLM/Data extractor 408) and determines a smallest number of tables that contains the data fields as column names by implementing one of the techniques of the data field mapper 416 described above. The query generator 410 determines the smallest number of tables that contains the data fields as column names by following a knowledge graph network architecture of the tables of the database, as described by the database schema, which can be represented in the metadata 414 of the database 412. The database schema (knowledge graph network architecture) describes the columns and tables of the database 412 and the corresponding relationship between each column and table of the database 412. Once the query generator 410 determines the smallest number of tables, it determines a shortest connected path among the tables with a data level join sequence (e.g., a subject data table joins to a site level data table first and then joins to a study level data table subsequently). In some cases, additional data bales are included in the joined data that correspond to unrequested but required data. From the joined data (i.e., a single data structure that includes all of the data from tables that include columns names that correspond to the data fields of the structured representation), the query generator 410 generates a standard database query (e.g., a SQL query) to access the data of the joined table. For example, a standard SQL template can be used, e.g., “select {columns} from {tables} where {joint condition} and {user requested filtering values};”.

To determine the smallest number of tables that contains the data fields of the structured representation as columns names, the query generator 410 can implement one or more rules. A first rule includes processing the user query 404 and an interpretation of the user query 404 by the LLM/Data Extractor 408. For example, for each data field of the structured representation, the query generator 410 can determine a data level (e.g., a data field of “country of a site” is a “site level data”, which is commonly found in clinical trial databases). As such, the query generator 410 can determine appropriate tables for the determined data level. For example, a “site country” data column may be included in a table named “AE count per subject” and in a table named “site info”. The “AE count per subject” table is a subject level table and the “site info” table is a site level table. As such, the query generator 410 can implement logic to determine the “site info” table to be a more relevant table for the “country of a site” data field because of the common data level between the data field and the identified table.

In addition, the query generator 410 can process a data table focus and/or theme to determine if a table is relevant to a particular data field. In some cases, based on the column names, the query generator 410 can determine a likely table focus (e.g., focused on site level data, study level data, or subject level data). In some cases, multiple tables of one or more databases satisfy matching criteria (e.g., matching column names, similar column names, matching level data, and matching table focus). In these cases, the query generator 410 can default to selecting tables from a shared database to reduce time and cost as compared to combining data from distinct databases.

In some implementations, table names, column names, and respective joints between tables as determined by the query generator 410 are stored in a database (e.g., database 412 or distinct database accessible by the query generator 410). In addition, the database can store other data and data structure characteristics including “table focus,” “table data level,” “column data level,” “meaning of data column,” and “data column formats” (e.g., text, numeric, date, etc.) for future use and faster analysis.

In some implementations, the query generator 410 accesses multiple databases with different database schema formats and other data structure formats. For example, some databases support “schema” and “DBlink”. In some cases, different business units of an organization have different schemas and DBlinks (e.g., set by a data management protocol) to access data. The query generator 410 can process relevant schemas and DBlinks and can store a “data access profile” for each database and can dynamically attach the schema and/or DBlink to the generated code, metadata 420, etc., to accommodate various formatting choices across an organization.

In some implementations, the database 412 includes one or more calculated tables that are derived from the data tables of the database 412. For example, a domain expert can generate a query to combine related data from multiple tables of the database 412 in a single table to be easily accessed by users and systems. In some cases, the calculated table is determined based on at least one rule defined by a domain expert.

A code generator 422 receives the dataset retrieved from the database 412 that is a subset of all the data in the database 412 based on the particular user query 404, to generate executable code for processing the received dataset. The executable code can be represented in a scripting language, e.g., Python. The executable code can include instructions for evaluating various characteristics of the received dataset. For example, the executable code can transform the dataset (e.g., normalization), determine high level statistical analysis (e.g., average, variance, etc.), or generate sub-datasets with various filters for further analysis and/or visualization (e.g., population analysis).

A code executor 424 can execute the executable code generated by the code generator 422. For example, the code executor 424 can include an execution environment, e.g., a Python execution environment, with necessary resources (memory, software packages, computing resources). An output of the code executor 424 can be transformed datasets, sub-datasets, statistical analysis, visualization instructions, or any other output derived from the output of the executable code.

The output of the code executor 424 is received by the chat UI 402. In some implementations, the output data is displayed on a data display 426 to be viewed and/or analyzed by a user. In some implementations, the data display 462 includes a tabular data display, a graphical data display, numerical indicators, and any other interface elements that facilitate the review and analysis of data.

The output of the code executor 424 is also received by the GenAI system 406, in which an output evaluator 428 can evaluate the output of the code executor 424. In some implementations, the evaluation is executed by an LLM of the GenAI system 406. In addition to the output of the code executor 424, the output evaluator 428 receives the user query 404. The output evaluator 428 interprets the overall goal of the user query 404. In addition, the output evaluator 428 can suggest a chart to visualize the data from the code executor 424 according to the interpreted goal of the user query 404. For example, the output evaluator 428, by generating outputs from the LLM, can suggest variables for the horizontal axis, vertical axis, chart colors, and suggested titles/axis labels of the suggested chart.

In some implementations, the output of the executable code (e.g., tabular data or chart) is displayed on the chat UI 402 and can be downloaded via a download link. Similarly, the output of the output evaluator 428 can be downloaded and/or viewed via a customizable chart or a data file (e.g., PPT, PDF, etc.).

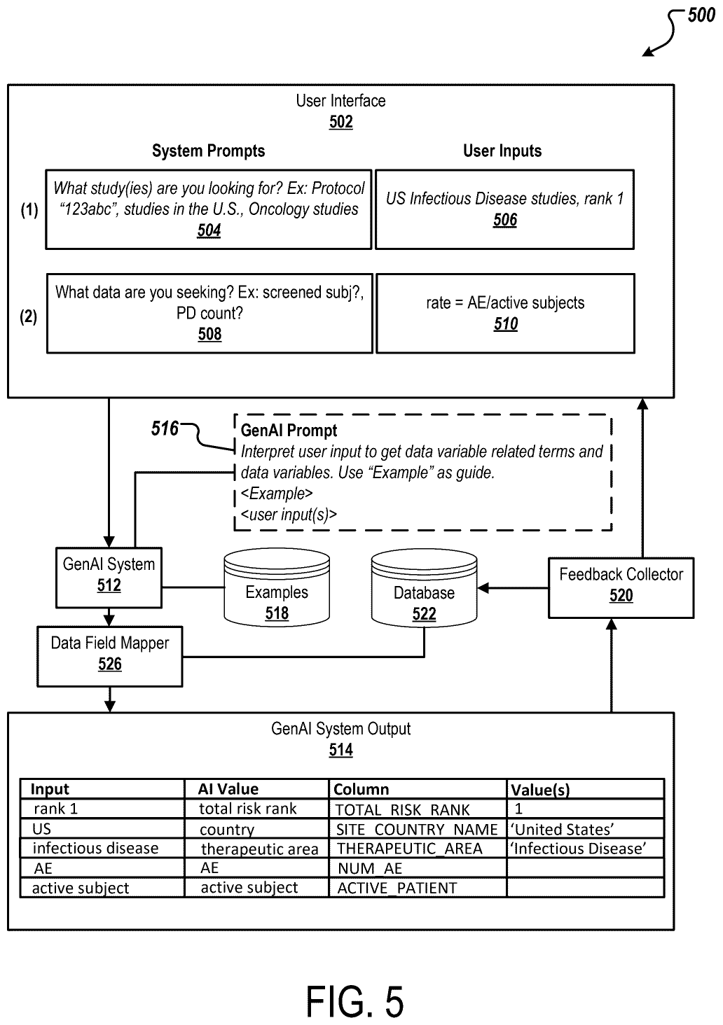

FIG. 5 illustrates an example system 500 for interpreting a user query with a GenAI system 512. The system 500 includes a user interface 502. In some implementations, the user interface 502 includes a chat interface. In some other implementations, the user interface 502 includes user input fields, in which a user inputs answers to particular questions. In both implementations, the GenAI system 512 receives one or more snippets of text received from the user.

For the example system 500, consider a GenAI system 512 that interacts with the user interface 502 by displaying the user specific prompts. A first prompt 504 states, “What study(ies) are you looking for? Ex: Protocol “123abc”, studies in the U.S., Oncology studies.” The user responds with a first user input 506: “US Infectious Disease studies, rank 1”. A second prompt 508 states, “What data are you seeking? Ex: screened subj?, PD count?.” The user responds with a second user input 510 to the second prompt 508: “rate=AE/active subjects”. This example includes a response (the second user input 510) from the user that specifies a particular equation for the system to evaluate when delivering the requested insights.

In some implementations, the user inputs 504, 506 are generated by an LLM of the GenAI system 512. In some implementations, the LLM of the GenAI system 512 processes domain-specific data, examples, and domain expertise to deliver relevant follow up questions.

The user inputs (e.g., user inputs 506, 510) are received by the GenAI system 512. The GenAI system accesses a database of examples 518. The data stored in the database of examples 518 include example user inputs and corresponding reviewed AI interpretations of the respective user input. For example, the database of examples 518 can include an entry, in which the entry includes a user input: “rank 1 sites in US, hematology studies,” and an AI interpretation: “rank 1=total risk rank: 1; US=country: US; hematology=therapeutic area: hematology.” The GenAI system 512 can identify a relevant example (through string similarity, embedded vector overlap, etc.) to use in a GenAI prompt 516.

The GenAI prompt 516 states: “Interpret user input to get data variable related terms and data variables. Use “Example” as guide. <Example><user input(s)>.” The GenAI prompt 516 includes an instruction for the present task, the identified example from the database of examples 518, and the received user inputs from the user interface 502 (e.g., user inputs 506, 510).

The GenAI system 512 processes the GenAI prompt 516 that includes instructions, the retrieved example, and the user inputs to generate a structured representation of the user inputs, as described in detail in the description related to the previous figures.

A data field mapper 526 processes the structured representation and determines columns names and/or tables names of a database 522 that match the identified data fields of the structured representation. For example, an example GenAI system output 514 illustrates the input variables identified by the GenAI system 512 as “rank 1”, “US”, “infectious disease”, “AE”, and “active subject.” Each of the identified variables correspond to words/phrases present in the user inputs 506, 510. The GenAI system output 514 also illustrates the AI values of column names predicted by the GenAI system 512. In other words, the AI values correspond to a “best guess” of a column name that corresponds to the identified variables. The AI values include “total risk rank”, “country”, “therapeutic area”, “AE”, and “active subject”.

The data field mapper 526 determines columns names of the database 522 that match the AI values, as represented in the GenAI system output 514 column “AI Value.” In some implementations, each column of the database 522 includes one or more alternative column names. In this case, the data field mapper 526 also matches each of the AI values with each of the alternative column names for each column of the database 522. The matched columns, as determined by the data field mapper 526 are illustrated in the GenAI system output 514 “Column” column. The identified database column names are “TOTAL_RISK_RANK”, “SITE_COUNTRY_RANK”, “THERAPEUTIC AREA”, “NUM_AE”, “ACTIVE PATIENT”.

The system 500 can implement a feedback collector 520 via a feedback user interface. In some implementations, the feedback user interface is the same as the user interface 502. In some other implementations, the feedback user interface is a separate user interface, in which a set of authorized personnel have access to review the GenAI system output 514 and modify one or more parameters of the GenAI system 512, database of examples 518, and database 522. The feedback collector 520 collects feedback indicative of the accuracy of the match between the “AI Value” column and the “Column” column of the GenAI system output 514. If there is an incorrect match, a user can edit the output and provide the correct column name. The feedback collector 520 can initiate an update of the database 522 to include the correct column name as an alternative column name of the respective column. In addition, a user of the user interface 502 and/or the feedback user interface can review the feedback collected by the feedback collector 520.

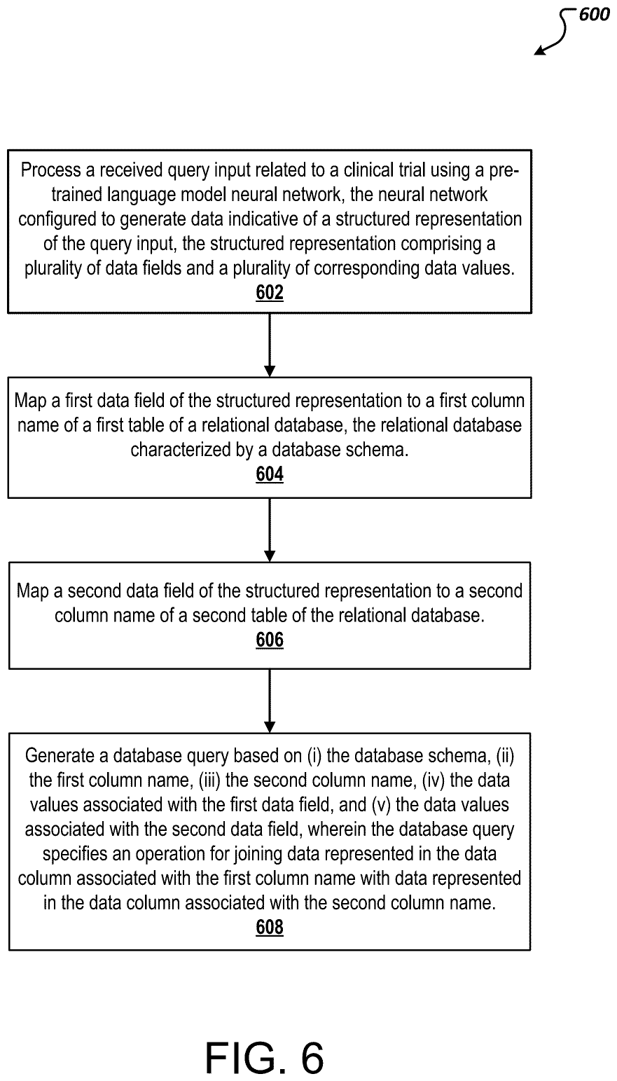

FIG. 6 is a flow diagram of an example process 600 for accessing clinical trial data. The process can be performed by a system similar to the system 100, which can include one or more computer systems.

The system processes (602) a received query input related to a clinical trial using a pre-trained language model neural network. The neural network is configured to generate data indicative of a structured representation of the query input. The structured representation includes multiple data fields and corresponding data values.

The system maps (604) a first data field of the structured representation to a first column name of a first table of a relational database, the relational database characterized by a database schema. In some implementations, the relational database includes at least one calculated table, in which the calculated table includes data from at least two tables of the relational database. In some implementations, the calculated table is determined based on one or more rules associated with received domain expertise. The system maps (606) a second data field of the structured representation to a second column name of a second table of the relational database.

In some implementations, the mapping of data fields of the structured representation to columns names of the relational database includes generating embedded representations of a data field of the structured representation and of at least a portion of a column name associated with a table of the relational database. In some implementations, the system determines a similarity metric between the data field and the portion of the column name, the similarity metric based on an overlap of the embedded representations. Furthermore, in some implementations, at least one column of at least one table of the relational database is characterized by a corresponding one alternative column name, the alternative column name different from the column name.

In some implementations, the system receives feedback indicative of an accuracy of the mapping of the first data field to the first column name and updates at least one alternative column name of a column of a table of the relational database.

The system generates (608) a database query based on (i) the database schema, (ii) the first column name, (iii) the second column name, (iv) the data values associated with the first data field, and (v) the data values associated with the second data field, wherein the database query specifies an operation for joining data represented in the data column associated with the first column name with data represented in the data column associated with the second column name.

In some implementations, the system executes the generated database query, in which the output of the executed query is a resulting data table that includes data values related to the input query. In some implementations, the resulting data table is displayed on a user interface as a visual representation of the resulting data table. In some implementations, a pre-trained neural language model neural network generates executable code (e.g., Python code) for analyzing the resulting data table (e.g., calculating statistical evaluations, generating advanced visual representations, initiating a communication of the resulting data table, etc.).

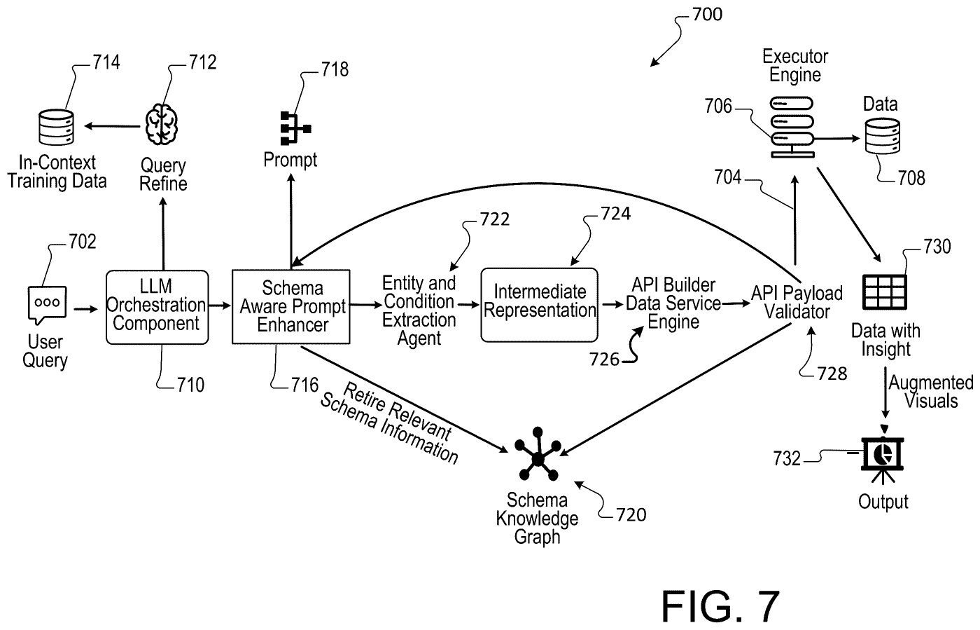

FIG. 7 illustrates a system 700 for generating a database query 704 based on a user query 702. The system 700 is particularly well suited for accessing and generating insights from data stored in a database 708 that is a localized database. In other words, the database 708 can be described by a set of data fields (e.g., column names) and data values that are specific to a particular data context (e.g., a particular company or application). In some cases, the data fields and/or data values are non-standard words or sequences of characters specific to a particular context (e.g., company, industry, clinical trial, etc.). Individuals closely associated with the particular context would likely be able to interpret the data fields and/or data values; however, a general foundational model is unlikely to correctly interpret the meaning of the data fields and/or data values because they are not represented in standard training data sets.

An LLM orchestration component 710 receives the user query 702. The LLM orchestration component 710 serves as a control system that controls a flow of data between various components of the system 700. For example, the LLM orchestration component 710 provides the user query 702 to be processed by LLMs of an entity and condition extraction agent 722, extracts data from relevant databases (e.g., a localized database or an in-context training data database 714), and initiates data processing tasks by a query refiner 712. In some implementations, the LLM orchestration component 710 is configurable to implement one or more data processing workflows that include one or more databases and one or more computing resources of the system 700. In some implementations, a new data processing step is added to an existing data processing workflow (e.g., access to conversation history, integration to external data through APIs and/or vector databases, and access to cached data). The orchestration component 710 can coordinate an implementation of the new data processing step by including the new functionality into a new or existing data processing workflow.

The query refiner 712 is operable as an intermediary between data retrieval systems (e.g., a system for retrieving data from a database like the in-context training data database 714) and an LLM (e.g., an LLM of the entity and condition extraction agent 722). The query refiner 712 implements data processing tasks to optimize for task-specific performance. For example, the query refiner 712 can perform one or more of (i) content extraction and restructuring, (ii) context alignment, (iii) token optimization, and (iv) plug-and-play integration.

Regarding (i), the query refiner 712 extracts query-relevant content from retrieved data from the in-context training data database 714 and sorts the retrieved data based on a hierarchy to highlight key information present in the retrieved data. The hierarchical sorting addresses various known data representation challenges including a “lost-in-the-middle” syndrome, in which scattered key information is overlooked.

Regarding (ii), the query refiner 712 performs data processing tasks that ensure that refined data (e.g., processed data) is contextually aligned with original data retrieved from a database (e.g., original data retrieved from the in-context training data database 714). In some implementations, the query refiner 712 executes data processing tasks to preserve verbatim details and data/text structure related to sections of data (e.g., bullet points, headers, chapters, etc.).

Regarding (iii), the query refiner 712 compresses and restructures data retrieved from a database (e.g., the in-context training data database 714). By compressing and restructuring retrieved data, the query refiner 712 reduces token usage by an LLM (e.g., the LLM of the entity and condition extraction agent 722), which reduces energy consumption and processing times.

Regarding (iv), the query refiner 712 processes data retrieved by RAG systems by including retrieved data in prompts to increase accuracy and relevance of outputs generated by LLMs of the system 700.

In some implementations, the in-context training data database 714 includes example prompt data from previous implementations of the system 700. In some implementations, the data stored in the in-context training data database 714 provides localized data and is processed by the query refiner 712 to modify an LLM prompt and the user query 702 to improve the ability of an LLM to generate relevant responses to the user query 702.

In some implementations, the in-context training data database 714 includes data representative of example user queries, expected data extracted from the corresponding example user queries, and text-based instructions of data post-processing steps. An example data entry stored in the in-context training data database 714 can be represented in a structured format as: {“user_query”: “report # of confirmed and planned CRA visits, study XXX”, “expected_extraction”: [“num of confirmed and planned CRA visits”-> (num of confirmed interim per study), “num of confirmed and planned CRA visits”-> (num of planned interim per study”, “no mention of future CRA site visit time”-> (scheduled CRA visit time, (now, next 12 months)), “study XXX”-> “study ID: (XXX)”], “brief_instruction”: “return a table showing study ID, num of confirmed visits, and num of planned visits [[for study XXX]]”}. The query refiner 712 can implement a string similarity (e.g., cosine similarity) between the user query 702 and data entries of the in-context training data database 714 to identify relevant examples to include in a prompt to an LLM. The data entries like the above example provide a mapping of key phrases within a user query to data variables. By processing a particular user query 702 along with a similar example extracted from the in-context training data database 714, an LLM is more likely to provide a relevant and accurate output data structure in response to the user query 702. A schema-aware prompt enhancer 716 processes the user query 702 and schema information from a schema knowledge graph 720 related to a localized database (e.g., field names, possible data values, etc.). The prompt enhancer 716 generates a prompt 718 that includes the user query 702 and schema information related to the localized database, which is processed by an LLM. In some cases, the LLM is trained with training data that does not include information about the data stored in the localized database, so providing the information in the prompt 718 allows the LLM to generate outputs within the context of the localized database.

The entity and condition extraction agent 722 processes the schema information from the schema knowledge graph 720 and the user query 702 to identify entities in the user query 702. In some implementations, the entity and condition extraction agent 722 implements syntactic entity extraction. Syntactic entity extraction implements fuzzy and/or exact search methods to identify local context entities (e.g., named entities in the user query 702 that are also present in the context of the localized database). In some implementations, the syntactic entity extraction utilizes time-period extraction to identify time periods in the user query 702 (e.g., last year, the last five days, etc.).

In some implementations, the syntactic entity extraction is a keyword-based lexical search technique that includes a retrieval of information from one or more databases by matching terms of the user query 702 with words and/or phrases in text data stored in a database or document index. In some cases, the syntactic entity extraction matches terms based on an exact match or a near-exact match. In some other cases, the syntactic entity extraction matches terms based on a similarity metric with a particular threshold value. In a general sense, the syntactic entity extraction only processes data based on text similarity (word matching) and does not consider semantic meaning of words and phrases in the user query 702 and in text data stored in databases and document indices. The syntactic entity extraction enables the system 700 to process localized data entities (e.g., terms specific to a particular data context) in comparison with LLM-based systems that rely on general knowledge contained in training data used to train the LLMs of the systems.

In addition to the syntactic entity extraction, in some implementations, the entity and condition extraction agent 722 implements semantic entity extraction that utilizes LLMs to identify entities in the user query 702. In some implementations, the semantic entity extraction includes invoking an LLM and providing it with the entities generated by the syntactic entity extraction to generate a database query (e.g., SQL query) that includes the identified entities in addition to other entities that may be identified by the LLM and not identified by the syntactic entity extraction method.

An example of the user query 702 is, “Show me sales in USD, product names, manufacturer and regions with sales greater than 1000000 in 2023 with active ingredient L4B1.” The example user query includes a context-specific data value (L4B1) in addition to a time period (2023). In some cases, the syntactic entity extraction process of the entity and condition extraction agent 722 identifies the following entities: {Entities_Dimension: {ATC4: L4B1}, Time_Period: {Year: 2023}}. The identified entities include an ATC4 chemical (Anatomical Therapeutic Chemical 4^{th} Level) of L4B1. This identified entity is an example of a data field and data value that is likely not to be represented in a training dataset of the LLM.

Furthermore, in relation to the example user query, the semantic entity extraction process can generate components of a SQL query that include {Entities_Dimensions: {Product, Manufacturer, Region}, Measures: {USD}, Filter: {USD>1000000}}. Taking into account the outputs from both the semantic entity extraction and the syntactic entity extraction, an intermediate representation 724 includes a combined representation that can be determined to be {Entities_Dimensions: [Product, Manufacturer, Region, ATC4, Time_Period], Entities_Measures: [USD], Filter: [ATC4: L4B1, USD>1000000], Time_Period Filter: {Type: Year, value: 2023}}, in which the intermediate representation 724 includes outputs from both the syntactic and semantic entity extraction processes.

A query builder data service engine 726 processes the intermediate representation 724 (e.g., a JSON data structure that includes elements of a database query like dimensions, measures, and filters). In some implementations, the query builder data service engine 726 implements a rule-based system for converting the intermediate representation 724 into a database query. The query builder data service engine 726 converts the intermediate representation 724 into a syntax error-free database query (e.g., SQL query) that represents the data and conditions of the intermediate representation 724 and is formatted to query a database type consistent with the localized database.

The query builder service engine 726 accepts a payload (e.g., an input data object) that includes parameters that define a process for converting the intermediate representation 724 to a data baes query. The query builder service engine 726 implements a deterministic rule-based process. In some implementations, the payload includes configuration data values including data source, table dimensions, data column names, metric names, measure names, time-periods, period-index, among others. The query builder service engine 726 can process the payload data values to generate database queries that are executed to retrieve data from a source database.

A query payload validator 728 processes the database query generated by the query builder data service engine 726. The query payload validator 728 accesses the schema knowledge graph 720 to confirm that the database query database query generated by the query builder data service engine 726 includes data fields (e.g., column names) that are represented in the localized database (e.g., the database 708). In some implementations, the query payload validator 728 validates the payload received by the query builder service engine 726 to confirm that the provided data values abide by a particular user's access control settings, data source availability, and logical accuracy before executing database queries on the source database. The query payload validator 728 validates the payload data values based on information stored in the schema knowledge graphs 720 and system-level information related to user roles (e.g., security and access information).

The executer engine 706 processes the validated database query from the query payload validator 728 and queries the database 708. The executer engine 706 receives a subset of data from the database 708 that is consistent with the database query 704. In some implementations, the data received from the database 708 is represented as a data table 730 and presented on a user interface 732.

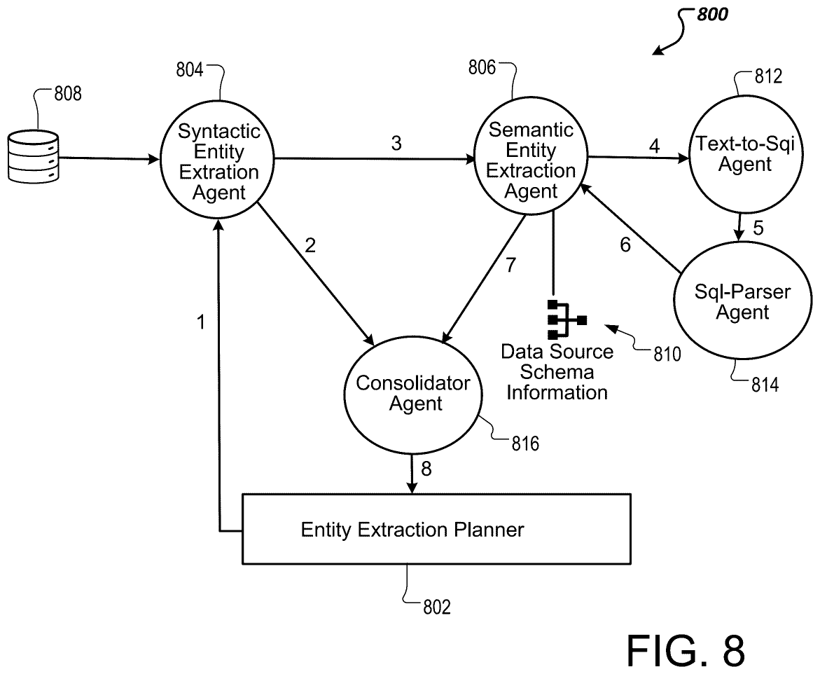

FIG. 8 illustrates a system 800 for generating a database query for a localized database. The system 800 includes an entity extraction planner 802 that orchestrates an order of operations to be implemented with respect to entity extraction.

The system 800 includes a syntactic entity extraction agent 804 and a semantic entity extraction agent 806. In some implementations, the syntactic entity extraction agent 804 accesses a local data configuration database 808 that includes data pertaining to the localized database. For example, the local data configuration database 808 can include data schema information. The data schema information can include data fields, data values, and data types that represent data stored in the localized database. The syntactic entity extraction agent 804, as described in relation to the entity and condition extraction agent 722 of FIG. 7, processes a user query in addition to the data stored in the local data configuration database 808 to extract relevant entities as they relate to data stored in the localized database.

In some implementations, the entity extraction planner 802 configures the semantic entity extraction agent 806 to process the user query in addition to the output of the syntactic entity extraction agent 804 to generate the database query. The semantic entity extraction agent 806 also accesses data source schema information 810 related to the localized database. The data source schema information 810 includes data fields and data types related to the data stored in the localized database.

In some implementations, a text-to-sql agent 812 processes the entities identified by the semantic entity extraction agent 806 to convert the identified entities into a SQL database query. In some other implementations, alternative text-to-database agents are implemented to convert the identified identities into database queries associated with alternative database environments (e.g., Postgres, NoSQL, etc.).

A sql-parser agent 814 processes the generated database query from the text-to-sql agent 812 and parses the database query to identify particular properties of the database query. For example, in the case of a SQL query, the sql-parser agent 814 identifies a “SELECT” statement, a “WHERE” statement, and a “GROUP BY” statement of the database query. In some cases, the text-to-sql agent 812 is implemented as an LLM, which can generate syntax errors in a generated database query. The sql-parser 814 extracts relevant statements from the generated database query into a structured format (e.g., JSON format).

A consolidator agent 816 receives the parsed database query generated by the semantic entity extraction agent 806 and the entities identified by the syntactic entity extraction agent 804 and combines the two outputs into a single database query. In some cases, the syntactic entity extraction agent 804 identifies entities in the user query that are specific to a particular data environment (e.g., company database, industry-specific terms, etc.), in which the data fields represented in the particular data environment are unlikely to be represented in training data used to train a general foundational model (e.g., commonly available LLMs). In some cases, the semantic entity extraction agent 806 identifies common data fields that are highly represented in LLM training data (e.g., country filters, common medications, demographic filters, etc.).

The entity extraction planner 802 receives the output of the consolidator agent 816. The operations executed by the entity extraction planner 802 are similar to the operations executed by the entity and condition extraction agent 722 of FIG. 7. The consolidator agent 816 consolidates extracted entities and passes the consolidated output downstream to generate an intermediate representation (e.g., the intermediate representation 724 of FIG. 7) to be received by a query builder service engine (e.g., the query builder service engine 726 of FIG. 7).

In some cases, a GenAI system that implements an LLM can exhibit hallucinations with respect to generated outputs. Hallucinations include generated outputs that include incorrect data, often represented as correct data. In some implementations, a GenAI system that implements default LLMs (e.g., an LLM that is trained on general training data) exhibit higher hallucination rates in comparison a GenAI system that is implemented with an LLM with access to a localized database. The table below illustrates a comparison of hallucination rates between a GenAI system that implements a default LLM with a GenAI system that implements an LLM with access to a localized database. As evident by the data represented in the table below, the LLM with localized data access exhibits a smaller hallucination rate. The evaluation that generated the data displayed in the table below included processing 43 user questions by each type of GenAI system.

| | | LLM with | Actual |
|---|---|---|---|
| | Default LLM | Localized Data | Entities |
| Total Detected Entities | 121 | 155 | 165 |
| Total Detected Entities (%) | 73.33% | 93.93% | |
| Hallucinations | 28 | 2 | |
| Hallucinations (%) | 16.40% | 1.20% | |

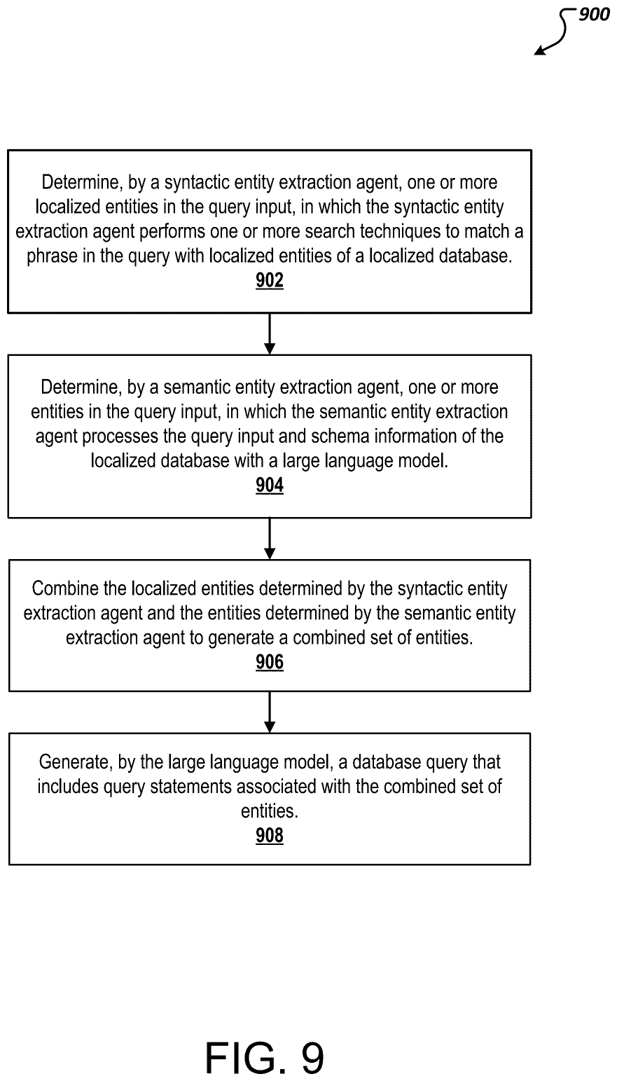

FIG. 9 is a flow diagram of an example process 900 for generating a database query based on a user query. The process can be performed by a system similar to the system 100, which can include one or more computer systems.

The system determines (902), by a syntactic entity extraction agent, one or more localized entities in the query input, in which the syntactic entity extraction agent performs one or more search techniques to match a phrase in the query with localized entities of a localized database. In some implementations, the search techniques include exact string matching and/or fuzzy string matching.

The system determines (904), by a syntactic entity extraction agent, one or more entities in the query input, in which the semantic entity extraction agent processes the query input and schema information of the localized database with a large language model. In some implementations, the semantic entity extraction agent processes the output of the syntactic entity extraction agent with a large language model to generate a database query. In some implementations, the schema information includes data field names, data field values, and data types pertaining to the data stored in the localized database.

The system combines (906) the localized entities determined by the syntactic entity extraction agent and the entities determined by the semantic entity extraction agent to generate a combined set of entities.

The system generates (908), by the large language model, a database query that includes query statements associated with the combined set of entities. In some implementations, the system generates the database query with one or more rules-based processes. In some implementations, the system parses the generated database query to determine associated database query statements. The statements can include select parameters, filter parameters, and grouping parameters. In some implementations, the system generates a final database query with the rules-based approach based on the determined database query statements and determines a subset of data from the localized database by querying the localized database with the generated final database query. In some implementations, the system displays the determined subset of data on a user interface.

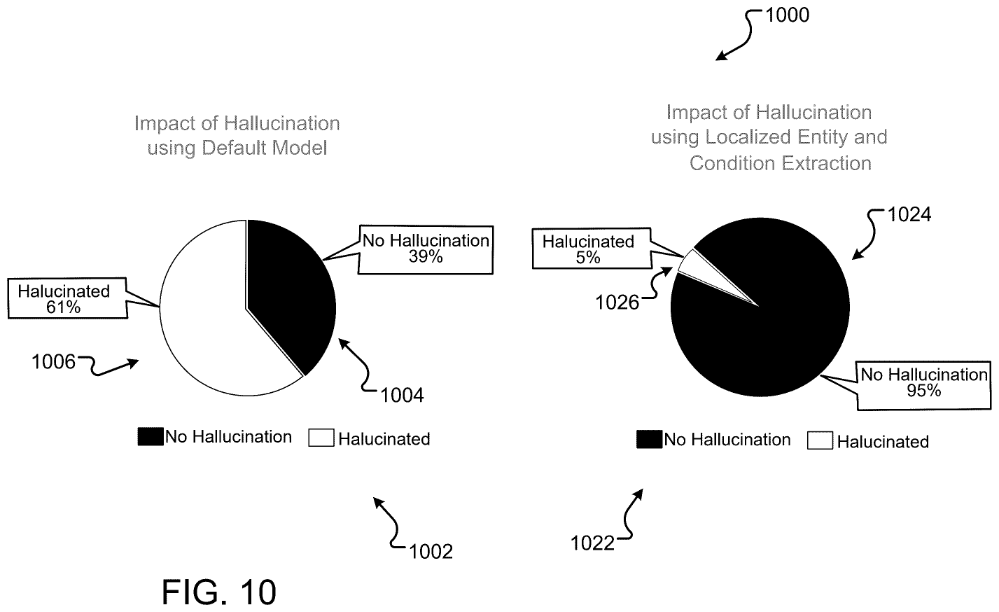

FIG. 10 is a graphical representation 1000 of a comparison between an implementation of a default GenAI system and a GenAI system that accesses a localized database. The representation 1000 includes a first pie chart 1002 that displays data associated with generated outputs of a default GenAI system (e.g., a system that implements an LLM without access to external databases, fine-tuning, or any other application-specific adjustments). The representation includes a second pie chart 1022 that displays data associated with generated outputs of a GenAI system that accesses a localized database. The localized database includes data associated with a particular entity, organization, or environment. The pie charts 1002, 1022 represent data indicative of a percentage of generated outputs that include hallucinations. Hallucinations generated by a GenAI system include outputs that are incorrect, fabricated, or not related to any real-world data. In some cases, hallucinations occur because an LLM of a generic GenAI system is trained on a broad set of generalized training data that does not include extensive data about particular data contexts.

The first pie chart 1002 includes a first graphical region 1004 that indicates the default GenAI system generates an output with a hallucination rate of 61% and a second graphical region 1006 that indicates the default GenAI system generates an output with a no-hallucination rate of 39%. The second pie chart 1022 includes a first graphical region 1024 that indicates the GenAI system with access to a localized database generates an output with a hallucination rate of 5% and a second graphical region 1026 that indicates the GenAI system with access to the localized database generates an output with a no-hallucination rate of 95%. As such, the GenAI system with access to the localized database exhibits improved performance in relation to the hallucination rates of generated outputs.

Embodiments of the subject matter and the functional operations described in this specification can be implemented in digital electronic circuitry, in tangibly-embodied computer software or firmware, in computer hardware, including the structures disclosed in this specification and their structural equivalents, or in combinations of one or more of them. Embodiments of the subject matter described in this specification can be implemented as one or more computer programs, i.e., one or more modules of computer program instructions encoded on a tangible non-transitory program carrier for execution by, or to control the operation of, data processing apparatus. Alternatively, or in addition, the program instructions can be encoded on an artificially-generated propagated signal, e.g., a machine-generated electrical, optical, or electromagnetic signal, that is generated to encode information for transmission to suitable receiver apparatus for execution by a data processing apparatus. The computer storage medium can be a machine-readable storage device, a machine-readable storage substrate, a random or serial access memory device, or a combination of one or more of them. The computer storage medium is not, however, a propagated signal.

The term “data processing apparatus” encompasses all kinds of apparatus, devices, and machines for processing data, including by way of example a programmable processor, a computer, or multiple processors or computers. The apparatus can include special purpose logic circuitry, e.g., an FPGA (field programmable gate array) or an ASIC (application-specific integrated circuit). The apparatus can also include, in addition to hardware, code that creates an execution environment for the computer program in question, e.g., code that constitutes processor firmware, a protocol stack, a database management system, an operating system, or a combination of one or more of them.

A computer program (which may also be referred to or described as a program, software, a software application, a module, a software module, a script, or code) can be written in any form of programming language, including compiled or interpreted languages, or declarative or procedural languages, and it can be deployed in any form, including as a stand-alone program or as a module, component, subroutine, or other unit suitable for use in a computing environment. A computer program may, but need not, correspond to a file in a file system. A program can be stored in a portion of a file that holds other programs or data, e.g., one or more scripts stored in a markup language document, in a single file dedicated to the program in question, or in multiple coordinated files, e.g., files that store one or more modules, sub-programs, or portions of code. A computer program can be deployed to be executed on one computer or on multiple computers that are located at one site or distributed across multiple sites and interconnected by a communication network.

Computers suitable for the execution of a computer program include, by way of example, can be based on general or special purpose microprocessors or both, or any other kind of central processing unit. Generally, a central processing unit will receive instructions and data from a read-only memory or a random access memory or both. The essential elements of a computer are a central processing unit for performing or executing instructions and one or more memory devices for storing instructions and data. Generally, a computer will also include, or be operatively coupled to receive data from or transfer data to, or both, one or more mass storage devices for storing data, e.g., magnetic, magneto-optical disks, or optical disks. However, a computer need not have such devices. Moreover, a computer can be embedded in another device, e.g., a mobile telephone, a personal digital assistant (PDA), a mobile audio or video player, a game console, a Global Positioning System (GPS) receiver, or a portable storage device, e.g., a universal serial bus (USB) flash drive, to name just a few.

Computer-readable media suitable for storing computer program instructions and data include all forms of non-volatile memory, media and memory devices, including by way of example semiconductor memory devices, e.g., EPROM, EEPROM, and flash memory devices; magnetic disks, e.g., internal hard disks or removable disks; magneto-optical disks; and CD-ROM and DVD-ROM disks. The processor and the memory can be supplemented by, or incorporated in, special purpose logic circuitry.

Embodiments of the subject matter described in this specification can be implemented in a computing system that includes a back-end component, e.g., as a data server, or that includes a middleware component, e.g., an application server, or that includes a front-end component, e.g., a client computer having a graphical user interface or a Web browser through which a user can interact with an implementation of the subject matter described in this specification, or any combination of one or more such back-end, middleware, or front-end components. The components of the system can be interconnected by any form or medium of digital data communication, e.g., a communication network. Examples of communication networks include a local area network (“LAN”) and a wide area network (“WAN”), e.g., the Internet.

While this specification contains specific implementation details, these should not be construed as limitations on the scope of any invention or of what may be claimed, but rather as descriptions of features that may be specific to particular embodiments of particular inventions. Certain features that are described in this specification in the context of separate embodiments can also be implemented in combination in a single embodiment. Conversely, various features that are described in the context of a single embodiment can also be implemented in multiple embodiments separately or in any suitable subcombination. Moreover, although features may be described above as acting in certain combinations and even initially claimed as such, one or more features from a claimed combination can in some cases be excised from the combination, and the claimed combination may be directed to a subcombination or variation of a subcombination.

Similarly, while operations are depicted in the drawings in a particular order, this should not be understood as requiring that such operations be performed in the particular order shown or in sequential order, or that all illustrated operations be performed, to achieve desirable results. In certain circumstances, multitasking and parallel processing may be advantageous. Moreover, the separation of various system modules and components in the embodiments described above should not be understood as requiring such separation in all embodiments, and it should be understood that the described program components and systems can generally be integrated together in a single software product or packaged into multiple software products.

Particular embodiments of the subject matter have been described. Other embodiments are within the scope of the following claims. For example, the actions recited in the claims can be performed in a different order and still achieve desirable results. As one example, the processes depicted in the accompanying figures do not necessarily require the particular order shown, or sequential order, to achieve desirable results. In certain implementations, multitasking and parallel processing may be advantageous.

1-20. (canceled)

21. A system for retrieving data from at least one database, the system comprising:
at least one processor; and
a memory storing instructions that, when executed by the at least one processor, cause the at least one processor to perform operations comprising:
determining, by a syntactic entity extraction agent, one or more localized entities in a received query input, wherein the syntactic entity extraction agent performs one or more search techniques to match a phrase in the query input with localized entities of a localized database;
determining, by a semantic entity extraction agent, one or more entities in the query input, wherein the semantic entity extraction agent processes the query input and schema information of the localized database with a large language model;
combining the localized entities determined by the syntactic entity extraction agent and the entities determined by the semantic entity extraction agent to generate a combined set of entities;
using the large language model to generate a database query that includes query statements associated with the combined set of entities; and
outputting data representative of the database query.

22. The system of claim 21, wherein:
the query input comprises natural language received in a user interface; and
outputting data representative of the database query comprises displaying the database query in the user interface.

23. The system of claim 21, the operations further comprising:
executing the generated database query, wherein an output of the executed database query is a resulting data table; and
displaying, on a user interface, a visual representation of the resulting data table.

24. The system of claim 21, the operations further comprising:
querying the localized database with the generated database query to determine a subset of data from the localized database.

25. The system of claim 24, the operations further comprising:
retrieving data indicative of user access settings, wherein the user access settings include data indicative of an authentication for displaying the determined subset of data on a user interface.

26. The system of claim 21, wherein:
the schema information of the localized database includes data field names, data field values, and data types pertaining to data stored in the localized database.

27. The system of claim 21, wherein:
the localized database includes data related to a clinical trial.

28. The system of claim 21, wherein:
the localized database includes a set of data fields and corresponding data values that are specific to a particular data context and are not represented in a standard training data set of a general foundational large language model.

29. The system of claim 21, the operations further comprising:
parsing the generated database query to determine associated database query statements, the statements comprising select parameters, filter parameters, and grouping parameters; and
generating a final database query with a rules-based approach based on the determined database query statements.

30. The system of claim 21, wherein the one or more search techniques comprise exact string matching and fuzzy string matching.

31. The system of claim 21, wherein the semantic entity extraction agent processes output of the syntactic entity extraction agent with the large language model to generate the database query.

32. The system of claim 21, wherein the entities determined by the semantic entity extraction agent are highly represented in training data used to train the large language model.

33. The system of claim 21, wherein the localized entities determined by the syntactic entity extraction agent are not highly represented in training data used to train the large language model.

34. A method for retrieving data from at least one database, the method comprising:
determining, by a syntactic entity extraction agent, one or more localized entities in a received query input, wherein the syntactic entity extraction agent performs one or more search techniques to match a phrase in the query input with localized entities of a localized database;
determining, by a semantic entity extraction agent, one or more entities in the query input, wherein the semantic entity extraction agent processes the query input and schema information of the localized database with a large language model;
combining the localized entities determined by the syntactic entity extraction agent and the entities determined by the semantic entity extraction agent to generate a combined set of entities;
using the large language model to generate a database query that includes query statements associated with the combined set of entities; and
outputting data representative of the database query.

35. The method of claim 34, wherein:
the query input comprises a natural language prompt received in a user interface; and
outputting data representative of the database query comprises displaying the database query in the user interface.

36. The method of claim 34, further comprising:
querying the localized database with the generated database query to determine a subset of data from the localized database.

37. The method of claim 36, further comprising:
retrieving data indicative of user access settings, wherein the user access settings include data indicative of an authentication for displaying the determined subset of data on a user interface.

38. The method of claim 34, wherein:
the localized database includes data related to a clinical trial.

39. The method of claim 34, wherein:
the entities determined by the semantic entity extraction agent are highly represented in training data used to train the large language model; and
the localized entities determined by the syntactic entity extraction agent are not highly represented in the training data used to train the large language model.

40. One or more non-transitory computer readable media storing instructions that, when executed by at least one processor, cause the at least one processor to retrieve data from at least one database by performing operations comprising:
determining, by a syntactic entity extraction agent, one or more localized entities in a received query input, wherein the syntactic entity extraction agent performs one or more search techniques to match a phrase in the query input with localized entities of a localized database;
determining, by a semantic entity extraction agent, one or more entities in the query input, wherein the semantic entity extraction agent processes the query input and schema information of the localized database with a large language model;
combining the localized entities determined by the syntactic entity extraction agent and the entities determined by the semantic entity extraction agent to generate a combined set of entities;
generating, using the large language model, a database query that includes query statements associated with the combined set of entities; and
outputting data representative of the database query.
